(19) United States
(12) Patent Application Publication
(10) Pub. No.: US 2012/0308814 A1
(43) Pub. Date: Dec. 6, 2012

(54) Method for Stabilizing Polyacrylate Pressure-Sensitive Adhesives in Admixture with Adhesive Resins

(75) Inventors: Manfred Spies, Bad Bramstedt (DE); Bernd Lühmann, Norderstedt (DE)

(73) Assignee: TESA SE, Hamburg (DE)

(21) Appl. No.: 13/515,110

(22) PCT Filed: Nov. 18, 2010
(86) PCT No.: PCT/EP2010/067732

§ 371 Date: Aug. 15, 2012

(30) Foreign Application Priority Data
Dec. 16, 2009 (DE) 10 2009 054 788.6

(51) Int. Cl. C09J 133/08; C07C 321/06; C08K 5/37; C09J 7/02

(52) U.S. Cl. 428/341; 428/355 AC; 428/354; 523/453; 568/51

(57) ABSTRACT

The invention relates to polyacrylate-based pressure-sensitive adhesives, containing at least one resin and at least one ortho, meta or para-cresol derivative, the aromatic ring thereof being substituted on at least two carbon atoms, wherein the substitutes are derivatives of thiols and/or thioethers. The invention further relates to adhesive tapes comprising at least one layer of said pressure-sensitive adhesive and to the use of said cresol derivatives as an antioxidant for polyacrylate pressure-sensitive adhesives.

The invention relates to a method for stabilizing polyacrylate pressure-sensitive adhesives in admixture with tackifier resins.

Polyacrylates based on acrylic esters with defined chain lengths of the ester side groups exhibit pressure-sensitive adhesive properties. If the length of the ester side groups goes beyond a certain level, the pressure-sensitive adhesive properties are lost, owing to the crystallization of the copolymer.

Polyacrylates display a wide-ranging profile of properties as compared with the other customary scaffold polymers for pressure-sensitive adhesives. The polyacrylate scaffold polymer has no double bonds (“saturated system”) and is therefore very stable with respect to aging processes. It is transparent, colorless, and proves to be extraordinarily stable even under UV exposure. On account of these circumstances, the addition of aging inhibitor or light stabilizer packages to polyacrylates is also not a mandatory necessity, in contrast, for example, to natural-rubber-based or styrene block copolymer-based pressure-sensitive adhesives.

Unsaturated compounds require protection from aging. In respect of the aging inhibitors, a fundamental distinction is made between primary and secondary antioxidants. The primary antioxidants intervene as chain terminators in the chain propagation step, whereas the secondary or preventive antioxidants destroy hydroperoxide groups which trigger chain initiation or chain branching.

The polyacrylates are exceeded in their aging characteristics by silicone pressure-sensitive adhesives, which, however, are significantly more expensive.

The possibility to adjust the desired technical profile of adhesive properties of pressure-sensitive adhesive polyacrylates only via the copolymer composition, however, is limited. For this reason, it is frequently necessary to add tackifying resins even to polyacrylate pressure-sensitive adhesives.

Resin components contemplated for polyacrylates include in principle both synthetic resins such as terpene-phenolic resins, for example, or else tackifier resins of the hydrocarbon type, and also natural resins such as rosin derivatives or polyterpenes in pure form or as a mixture of different resins, for example.

Polyacrylates are polar scaffold polymers. In order for a tackifier resin to exhibit tackifying activity effectively in an elastomeric scaffold polymer, it is necessary fundamentally for a number of criteria to be met. On the one hand, the tackifier resin must be compatible with the scaffold polymer. Furthermore, the glass transition point of the tackifier resin ought to lie significantly above the glass transition point of the elastomer. As a supplement to this, the molar mass of the tackifier resin ought to be much lower than the molar mass of the elastomeric scaffold polymer.

On the basis of these circumstances, terpene-phenolic resins, aromatically modified HC resins, and rosin derivatives, more particularly hydrogenated rosin derivatives, are in the focal point of applications, on account of the good aging characteristics.

All of these tackifier resins, however, have the drawback that the resins exhibit a more or less strong inherent color, which means that the addition of these resins to a transparently clear and completely uncolored pressure-sensitive adhesive results in a discoloration of the pressure-sensitive adhesive, and the colorlessly transparent quality of the polyacrylate pressure-sensitive adhesive is lost.

Resin-blended polyacrylates which are coated in solution on a carrier material and subsequently dried in a drying tunnel at higher temperatures experience a thermal load which is accompanied by degradation, resulting in a slight browning of the pressure-sensitive adhesive. The solvent-free coating of polyacrylate pressure-sensitive adhesives is gaining importance more and more, owing to the wide-ranging advantages of solvent-free operation, such as the possibility of coating in thick layers, for example. Where, however, a pressure-sensitive adhesive (PSA) is coated onto a carrier material via a hotmelt process with solvent-free coating technology, the PSA experiences an even higher thermal load. Temperatures of around 160° C. for periods of up to 30 minutes, and possibly even longer, are entirely customary. A thermal load of this kind results in oxidative damage to the added tackifier resins, and this is reflected, depending on the tackifier resin used, in a browning of the resin and hence in a browning of the resin-blended polyacrylate PSA.

The use of aging inhibitors of the thioalkyl-substituted cresol derivative type for resin-blended polyacrylate PSAs with the objective of stabilizing the color of the compound, has not been carried out to date in the art.

Aging inhibitors of this kind have been described in the past only in combination with double-bond-containing elastomers that are suitable for pressure-sensitive adhesive applications.

For instance, Schmierer et al. in US20080070053 and Miller et al. in WO2008036322 describe the use of 4,6-bis(dodecylthiomethyl)-o-cresol as an aging inhibitor in PSAs of high shear strength that are based on styrene block copolymers. Mader et al. in WO2006128799 use thioalkyl-substituted cresols for protection against photooxidative degradation in butadiene-containing copolymers. In WO2006095015, Dubois et al. use aging inhibitor packages with Irganox 1726 for PSAs based on different block copolymers and tackifier resins. In WO2006003092, Maeder et al. utilize 4,6-bis(dodecylthiomethyl)-o-cresol for stabilizing polyether polyols, polyester polyols, and polyurethanes.

It was an object of the invention to remedy this situation and to indicate a possible way of being able to prepare tackifier resin-blended polyacrylate PSAs which, following thermal load, more particularly following coating via a hotmelt process, continue to display a very light color, remain largely transparent, and do not suffer dark discoloration.

This object has been achieved through use of specific aging inhibitors which carry a primary and a secondary aging inhibitor function in unison in one molecule.

The invention relates accordingly to a polyacrylate-based pressure-sensitive adhesive comprising at least one resin and also at least one cresol derivative (ortho-, meta- and/or para-cresol derivative) whose aromatic ring is substituted on at least two carbon atoms by sulfur-containing substituents, the substituents being derivatives of thiols and/or thioethers, in other words having the building blocks—S—R and/or—R—S—R (referred to below as thioalkyl chains) if R stands for organic radicals, more particularly for hydrocarbon chains (the cresol compounds modified accordingly are also referred to below as thioalkyl-modified cresol derivatives).

The substitution of the aromatic ring of the cresol derivative by the aforementioned substituents takes place more particularly on at least two of the C3, C4, C5, and C6 carbon atoms of the aromatic ring.

The aging inhibitors of the invention are more particularly cresol derivatives whose aromatic ring is substituted in ortho- and meta-position to the OH group (hydroxyl group) by thioalkyl chains, it being possible for the sulfur atom to be joined directly (unmediatedly) or else indirectly, more particularly via one or more alkyl chains, to the aromatic ring of the cresol building block.

In a preferred way, the sulfur-containing substituents of the cresol derivative are selected from the group G1, encompassing—S—R and—(CH₂)ₓ—S—R, where x represents a whole number and the radicals R independently of one another represent organic radicals, being selected with particular preference from the group G3 of the branched and unbranched, saturated, aliphatic hydrocarbon groups (alkyl groups).

The number of carbon atoms between the aromatic and the sulfur atom is advantageously between 1 and 10, preferably between 1 and 4 (such that, more particularly, 1×10, more particularly x=1, 2, 3 or 4).

The number of carbon atoms of the alkyl side chains (alkyl groups R) is preferably in the range from 1 to 25, preferably in the range from 6 to 16. Particularly advantageous aging inhibitors are those in which the alkyl groups R are groups having 8, 9, 10, 11 and/or 12 carbon atoms.

In one very preferred way, the two or optionally more than two sulfur-containing substituents of the cresol derivative are selected identically.

Preferred cresol derivatives of the invention can be described by the formula

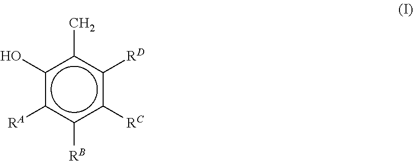

where two of the radicals R^{A}, R^{B}, R^{C}, and R^{D} are selected from the group G1, and the other two radicals R^{A}, R^{B}, R^{C}, and R^{D} are selected independently of one another from the group G2, encompassing branched and unbranched alkyl chains, and also hydrogen. More particularly the two radicals which are not sulfur-containing substituents are hydrogen. With further advantage, in this formula as well, the sulfur-containing substituents are located in ortho-position and in meta-position to the hydroxyl group (C6 and C4 positions).

Very preferred examples of aging inhibitors of the invention are the compounds of the 4,6-bis(alkylthioalkyl)-o-cresol type, as for example 4,6-bis(dodecylthiomethyl)-o-cresol, 4,6-bis(undecylthiomethyl)-o-cresol, 4,6-bis(decylthiomethyl)-o-cresol, 4,6-bis(nonylthio-methyl)-o-cresol and 4,6-bis(octylthiomethyl)-o-cresol. Aging inhibitors of these kinds are available, for example, from Ciba under the name Irganox® 1726 or Irganox® 1520.

The cresol-derivative aging inhibitors of the invention can be used alone (one or more of the cresol aging inhibitors) or in a mixture with other aging inhibitors, in which case other aging inhibitors used may preferably be sterically hindered phenols and/or UV absorbers.

The amount of the added aging inhibitor or—in the case of two or more aging inhibitors—of the added aging inhibitors is advantageously in a range between 0.1% and 10% by weight, preferably in a range between 0.2% and 5% by weight, more preferably in a range between 0.5% and 3% by weight, based on the overall solids content.

The polyacrylate PSAs (polyacrylate-based PSAs) are saturated systems, in other words polymers which have no double bonds or only an insignificant residual number of double bonds.

As tackifier resins it is possible to use the customary tackifier resins known from the prior art; advantageously, the at least one resin is selected from the group G4, encompassing terpene-phenolic resins, rosin derivatives, hydrocarbon resins having aromatic fractions, polyterpene resins, aromatically modified polyterpene resins, mixtures of the aforementioned resins.

The invention additionally provides for the use of cresol derivatives (ortho-, meta- and/or para-cresol derivatives) whose aromatic ring is substituted on at least two carbon atoms by sulfur-containing substituents, the substituents being derivatives of thiols and/or thioethers, as aging inhibitors for polyacrylate PSAs, more particularly for resin-containing polyacrylate PSAs. The cresol derivatives used in accordance with the invention as aging inhibitors for polyacrylate PSAs, more particularly for resin-containing polyacrylate PSAs, are in particular one or more of the cresol derivatives represented above, alone or in a mixture with other aging inhibitors, more particularly the other aging inhibitors identified.

### Experimental Investigations

Polyacrylate polymers with different compositions, based on 2-ethylhexyl acrylate, butyl acrylate, glycidyl methacrylate, and acrylic acid, in combination with various tackifier resins, especially terpene-phenolic resins, were investigated.

The following resins may be stated as examples:

| Resins: | Class of substance | Softening point (R&B): |
|---|---|---|
| DT110 (DRT) | terpene-phenolic resin | 115° C. |
| TP115 (Arizona) | terpene-phenolic resin | 115° C. |
| TP95 (Arizona) | terpene-phenolic resin | 95° C. |
| TF-100 (Xinyi) | terpene-phenolic resin | 100° C. |

In all cases the color of the polyacrylate PSAs used was stable in line with expectations. The tackifier resins used all have a more or less strong inherent color, which changed further to darker colors as a function of the storage temperature. Terpene-phenolic resins with a low softening point exhibited a greater tendency here to undergo dark discoloration under the influence of temperature.

Conventional primary or secondary antioxidants of the sterically hindered amine or phosphite or organic sulfide type did not exhibit any notable activities in the investigations conducted. Those investigated included 2,6-di-t-butyl-4-methylphenol (BHT), tetrakis(methylene 3-3,5-di-t-butyl-4-hydroxyphenyl)propionate)methane (Irganox 1010), 2,4-bis(n-octylthio)-6-(4-hydroxy-3,5-di-t-butylanilino)-1,3,5-triazine (Irganox565) or tris(dinonylphenyl) phosphite (Irgafos TNPP) or mixtures thereof.

The amounts used in this case were between 0.2% and 2% by weight, based on the solids content of the PSA.

In the course of the trials conducted it was found that the aging inhibitors known for unsaturated elastomers do not have a sufficiently stabilizing effect on the color of polyacrylate-tackifier resin compounds.

A very good effect in terms of stabilizing the color of the PSAs at relatively high temperatures was obtainable only with aging inhibitor from the group of the aging inhibitors of the invention, here more particularly 4,6-bis(alkylthioalkyl)-o-cresols, preferably Irganox® 1726 and Irganox® 1520.

Through use of the aging inhibitors of the invention it was possible to cause the resin-blended PSAs—even under thermal load—not to exhibit any tendency to become darker over time. In some cases it was in fact possible to observe a lightening of the PSAs through use of the aging inhibitors of the invention.

This makes it possible to use the resin-blended PSAs even in the hotmelt process, without any darkening of the PSAs. Accordingly it is possible in a simple way for resin-blended PSAs as well to be processed outstandingly in the hotmelt process, and for light-colored, transparent and/or colorless products to be produced.

The results of the investigations conducted are summarized in the table below.

Definitions here are as follows:

| Irganox | 4,6-bis(octylthiomethyl)-o-cresol |
|---|---|
| 1520 | (Irganox ® 1520; Ciba) |
| Irganox | 4,6-bis(dodecylthiomethyl)-o-cresol |
| 1726 | (Irganox ® 1726; Ciba) |
| DT110 | terpene-phenolic resin, softening point 115° C. |
| | (Dertophene ® T 110; DRT; softening point according to |
| | manufacturer: 111° C.) |
| TP115 | terpene-phenolic resin, softening point 115° C. |
| | (Sylvares ® TP 115, Arizona) |
| TP95 | terpene-phenolic resin, softening point 95° C. |
| (Arizona) | (Sylvares ® TP 95, Arizona) |
| TF-100 | terpene-phenolic resin, softening point 100° C. |
| | (TF-100 ®, Xinyi)) |
| Tinuvin | UV absorber; mixture of bis(1,2,2,6,6-pentamethyl-4- |
| 765 | piperidyl) sebacate and methyl 1,2,2,6,6-pentamethyl-4- |
| | piperidylsebacate (CAS 41556-26-7 and 82919-37-7); |
| | (Tinuvin ® 765, Ciba) |
| Irgafos | tris(2,4-di-tert-butylphenyl) phosphite (CAS 31570-04-4) |
| 168 | (Irgafos ® 168, Ciba) |
| Irgavos | tetrakis(2,4-di-tert-butylphenyl) 4,4-biphenyldiphosphonite |
| P-EPQ | (CAS 119345-01-6) |
| | (Irgafos ® P-EPQ, Ciba) |
| Desmodur | aliphatic polyisocyanate [low-viscosity hexamethylene |
| N3600 | diisocyanate(HDI) trimer] |
| | (Desmodur ® N3600, BayerMaterialScience) |
| Irganox | tetrakis(methylene-(3,5-di(tert)-butyl-4- |
| 1010 | hydrocinnamate))methane (CAS 6683-19-8) |
| | (Irganox ® 1010, Ciba) |
| BHT | butylated hydroxytoluene (=2,6-di-tert-butyl-p-cresol) (CAS |
| | 128-37-0) |

The names of some of the compounds identified are subject to brand protection. The omission of a corresponding mark in the table does not mean that the designations can be freely used.

The quantity figures in the table refer to parts by weight (TL).

Coat weight: 50 g/cm²

| | | | |
|---|---|---|---|
| No. | L* | a* | b* |

No thermal load (drying at RT)

| | | | | |
|---|---|---|---|---|
| 1 | 70TL Polyacrylate + 30TL DT110 | 90.35 | −0.86 | 9.03 |
| 2 | 70TL Polyacrylate + 30TL TP115 | 90.2 | −0.65 | 9.37 |
| 3 | 70TL Polyacrylate + 30TL TP95 | 30.21 | −0.76 | 9.61 |
| 4 | 70TL Polyacrylate + 30TL TF100 | 90.12 | −2.22 | 13.34 |
| 5 | 70TL Polyacrylate + 30TL TP95 + 1TL Irganox 1726 | 89.93 | −0.04 | 7.67 |
| 6 | 70TL Polyacrylate + 30TL TP95 + 1TL Irganox 1726 + 1TL Tinuvin 765 | 90.12 | 0.21 | 6.79 |
| 7 | 70TL Polyacrylate + 30TL TP95 + 1TL Irganox 1726 + 1TL Irgafos 168 | 90.4 | 0.27 | 6.34 |
| 8 | 70TL Polyacrylate + 30TL TP95 + 2TL Irganox 1726 | 90.45 | 0.31 | 6.21 |
| 9 | 70TL Polyacrylate + 30TL TP95 + 2TL Irganox 1726 + 2TL Tinuvin 765 | 90.42 | 0.36 | 6.21 |
| 10 | 70TL Polyacrylate + 30TL TP95 + 2TL Irganox 1726 + 2TL Irgafos 168 | 90.35 | 0.31 | 6.32 |
| 11 | 70TL Polyacrylate + 30TL TP95 + 0.5TL Irganox 1726 | 90.27 | 0.09 | 6.89 |
| 12 | 70TL Polyacrylate + 30TL TP95 + 0.5TL Irganox 1726 + 0.5TL Tinuvin 765 | 90.45 | 0.28 | 6.43 |
| 13 | 70TL Polyacrylate + 30TL TP95 + 0.5TL Irganox 1726 + 0.5TL Irgafos 168 | 90.36 | 0.08 | 6.93 |
| 14 | 70TL Polyacrylate + 30TL TP95 + 1TL Irganox 1726 + 1TL Irgafos P-EPQ | 90.31 | 0.13 | 7.11 |
| | Polyacrylate (reference) | 90.4 | 1.01 | 4.04 |

Standard drying 15 min 120° C.

| | | | | |
|---|---|---|---|---|
| 15 | 70TL Polyacrylate + 30TL DT110 | 90.22 | −0.88 | 9.29 |
| 16 | 70TL Polyacrylate + 30TL TP115 | 90.14 | −0.55 | 9.19 |
| 17 | 70TL Polyacrylate + 30TL TP95 | 90.07 | −0.7 | 9.65 |
| 18 | 70TL Polyacrylate + 30TL TF100 | 90.07 | −2.28 | 13.87 |
| 19 | 70TL Polyacrylate + 30TL TP95 + 1TL Irganox 1726 | 90.25 | 0.1 | 6.94 |
| 20 | 70TL Polyacrylate + 30TL TP95 + 1TL Irganox 1726 + 1TL Tinuvin 765 | 90.43 | 0.34 | 6.21 |
| 21 | 70TL Polyacrylate + 30TL TP95 + 1TL Irganox 1726 + 1TL Irgafos 168 | 90.33 | 0.28 | 6.43 |
| 22 | 70TL Polyacrylate + 30TL TP95 + 2TL Irganox 1726 | 90.51 | 0.37 | 6.09 |
| 23 | 70TL Polyacrylate + 30TL TP95 + 2TL Irganox 1726 + 2TL Tinuvin 765 | 90.43 | 0.45 | 6 |
| 24 | 70TL Polyacrylate + 30TL TP95 + 2TL Irganox 1726 + 2TL Irgafos 168 | 90.46 | 0.28 | 6.41 |
| 25 | 70TL Polyacrylate + 30TL TP95 + 0.5TL Irganox 1726 | 90.56 | 0.25 | 6.34 |
| 26 | 70TL Polyacrylate + 30TL TP95 + 0.5TL Irganox 1726 + 0.5TL Tinuvin 765 | 90.19 | 0.28 | 6.62 |
| 27 | 70TL Polyacrylate + 30TL TP95 + 0.5TL Irganox 1726 + 0.5TL Irgafos 168 | 90.44 | 0.15 | 6.75 |
| 28 | 70TL Polyacrylate + 30TL TP95 + 1TL Irganox 1726 + 1TL Irgafos P-EPQ | 90.31 | 0.1 | 7.31 |
| | Polyacrylate (reference) | 90.4 | 1.01 | 4.04 |
| | no thermal load | | | |

Additional thermal load 10 min at 140° C.

| | | | | |
|---|---|---|---|---|
| 29 | 70TL Polyacrylate + 30TL DT110 | 90.14 | −1.01 | 10.16 |
| 30 | 70TL Polyacrylate + 30TL TP115 | 89.86 | −0.59 | 10.04 |
| 31 | 70TL Polyacrylate + 30TL TP95 | 90.11 | −0.66 | 10.16 |
| 32 | 70TL Polyacrylate + 30TL TF100 | 89.68 | −2.33 | 15.54 |
| 33 | 70TL Polyacrylate + 30TL TP95 + 1TL Irganox 1726 | 90.32 | 0.17 | 6.76 |
| 34 | 70TL Polyacrylate + 30TL TP95 + 1TL Irganox 1726 + 1TL Tinuvin 765 | 90.38 | 0.28 | 6.65 |
| 35 | 70TL Polyacrylate + 30TL TP95 + 1TL Irganox 1726 + 1TL Irgafos 168 | 90.4 | 0.26 | 6.46 |
| 36 | 70TL Polyacrylate + 30TL TP95 + 2TL Irganox 1726 | 90.52 | 0.28 | 6.41 |
| 37 | 70TL Polyacrylate + 30TL TP95 + 2TL Irganox 1726 + 2TL Tinuvin 765 | 90.48 | 0.32 | 6.43 |
| 38 | 70TL Polyacrylate + 30TL TP95 + 2TL Irganox 1726 + 2TL Irgafos 168 | 90.39 | 0.22 | 6.65 |
| 39 | 70TL Polyacrylate + 30TL TP95 + 0.5TL Irganox 1726 | 90.52 | 0.1 | 6.8 |
| 40 | 70TL Polyacrylate + 30TL TP95 + 0.5TL Irganox 1726 + 0.5TL Tinuvin 765 | 90.35 | 0.26 | 6.58 |
| 41 | 70TL Polyacrylate + 30TL TP95 + 0.5TL Irganox 1726 + 0.5TL Irgafos 168 | 90.47 | 0.05 | 6.99 |
| 42 | 70TL Polyacrylate + 30TL TP95 + 1TL Irganox 1726 + 1TL Irgafos P-EPQ | 90.27 | −0.01 | 7.75 |
| | Polyacrylate (reference) | 90.4 | 1.01 | 4.04 |
| | no thermal load | | | |

Additional thermal load 3 hours at 140° C.

| | | | | |
|---|---|---|---|---|
| 43 | 70TL Polyacrylate + 30TL DT110 | 86.92 | −0.24 | 22.45 |
| 44 | 70TL Polyacrylate + 30TL TP115 | 85.89 | 0.65 | 23.87 |
| 45 | 70TL Polyacrylate + 30TL TP95 | 87.62 | −0.52 | 20 |
| 46 | 70TL Polyacrylate + 30TL TF100 | 85.86 | −0.6 | 28.87 |
| 47 | 70TL Polyacrylate + 30TL TP95 + 1TL Irganox 1726 | 89.42 | −0.61 | 11.68 |
| 48 | 70TL Polyacrylate + 30TL TP95 + 1TL Irganox 1520 | 87.92 | −0.62 | 11.92 |
| 49 | 70TL Polyacrylate + 30TL TP95 + 1TL Irganox 1726 + 1TL Tinuvin 765 | 88.87 | −0.02 | 12.31 |
| 50 | 70TL Polyacrylate + 30TL TP95 + 1TL Irganox 1726 + 1TL Irgafos 168 | 89.65 | −0.4 | 10.51 |
| 51 | 70TL Polyacrylate + 30TL TP95 + 2TL Irganox 1726 | 89.81 | −0.3 | 9.82 |
| 52 | 70TL Polyacrylate + 30TL TP95 + 2TL Irganox 1520 | 89.69 | −0.34 | 10.12 |
| 53 | 70TL Polyacrylate + 30TL TP95 + 2TL Irganox 1726 + 2TL Tinuvin 765 | 89.78 | −0.06 | 9.35 |
| 54 | 70TL Polyacrylate + 30TL TP95 + 2TL Irganox 1726 + 2TL Irgafos 168 | 89.41 | −0.39 | 12.51 |
| 55 | 70TL Polyacrylate + 30TL TP95 + 0.5TL Irganox 1726 | 88.38 | −0.03 | 14.53 |
| 56 | 70TL Polyacrylate + 30TL TP95 + 0.5TL Irganox 1726 + 0.5TL Tinuvin 765 | 88.63 | 0.07 | 12.77 |
| 57 | 70TL Polyacrylate + 30TL TP95 + 0.5TL Irganox 1726 + 0.5TL Irgafos 168 | 88.58 | −0.27 | 14.94 |
| 58 | 70TL Polyacrylate + 30TL TP95 + 1TL Irganox 1726 + 1TL Irgafos P-EPQ | 89.64 | −0.39 | 10.97 |
| 59 | 70TL Polyacrylate + 30TL TP95 + 1TL Irganox 1010 | 86.31 | −0.48 | 19.2 |
| 60 | 70TL Polyacrylate + 30TL TP95 + 2TL BHT | 90.34 | −0.42 | 18.94 |
| | Polyacrylate (reference) | 90.4 | 1.01 | 4.04 |
| | no thermal load | | | |

Determination of colors and gloss with BYK Gardner spectro-guide colorimeter. Colors determined on the CIELab scale with illuminant D65/10°.
L* = lightness (0 = black, 100 = white)
b* = yellow-blue (−120 = blue, +120 = yellow)
a* = red-green (−120 = green +120 = red)

Important for the assessment of yellowing is the color shift on the b* axis in the three-dimensional color space.

As can be seen from the tables, there is no notable change in the polyacrylate compounds after addition of 1% or 2% of Irganox 1726, even after temperature loading. With Irganox 1726 it is even possible to stabilize terpene-phenolic resins having relatively low softening points (95° C.), which in terms of their color stability under the influence of temperature (up to about 160° C.) are often less stable than terpene-phenolic resins having softening points above 110° C.

An increase in the concentration of Irganox 1726 has a stabilizing effect on the color of the compound. The example with Irganox 1520 shows this behavior as well.

### EXAMPLES

### Example 1

A polyacrylate PSA prepared by radical polymerization in 60/95 benzine spirit and consisting of a copolymer based on 48.5 mol % of 2-ethylhexyl acrylate, 48.5 mol % of butyl acrylate, 1 mol % of acrylic acid, and 2 mol % of glycidyl methacrylate, having a weight-average molar mass (M_{w}) of about 1.5 million g/mol, is compounded with about 30% by weight of the terpene-phenolic resin TP95 from Arizona. The compound is subsequently admixed with 2% by weight of Irganox 1726 in solution in 60/95 benzine spirit. The compound is subsequently homogenized on a roller bed for 24 hours.

The resulting resin-blended polyacrylate PSA is coated onto an etched PET film, with a coat weight of 50 g/m², using a coating knife in a laboratory coating unit.

The coated material is dried at room temperature for 24 hours and subjected to color measurement as a reference.

Subsequently the reference material is heat-treated at 150° C. in a force-air drying oven for 3 hours.

The color and the gloss of the differently stored adhesive tapes and also of the unstored adhesive tape were measured using a BYK Gardner spectro-guide colorimeter. The colors were determined on the CIELab scale with the illuminant D65/10°.

The definitions of the colors in the three-dimensional color space in question are as follows:
L*=lightness (0=black, 100=white)
a*=red-green (−120=green, +120=red)
b*=yellow-blue (−120=blue, +120=yellow)

For each sample, 3 measurements were performed at 3 different locations.

The reference used was a white tile (V+B Steinzeug, article No. 1106 TW01, color 45×). The specimens were subsequently adhered to a reference tile and subjected to measurement.

Results:

Colorimetry

| Specimen | Thermal load | L* | a* | b* |
|---|---|---|---|---|
| Polyacrylate adhesive | none | 30.21 | −0.76 | 9.61 |
| of Example 1 | | | | |
| Polyacrylate adhesive | 3 hours at 150° C. | 89.81 | −0.3 | 9.82 |
| of Example 1 | | | | |
| Polyacrylate adhesive | 3 hours at 150° C. | 87.62 | −0.52 | 20 |
| of Example 1 without | | | | |
| aging inhibitor | | | | |

### Example 2

A polyacrylate PSA prepared by radical polymerization in 60/95 benzine spirit and consisting of a copolymer based on 48.5 mol % of 2-ethylhexyl acrylate, 48.5 mol % of butyl acrylate, 1 mol % of acrylic acid, and 2 mol % of glycidyl methacrylate, having a weight-average molar mass (M_{w}) of about 1.5 million g/mol, is compounded with about 30% by weight of the terpene-phenolic resin TP95 from Arizona. The compound is subsequently admixed with a mixture of 1% by weight of Irganox 1726 and 1% by weight of Irgafos 168 in solution in 60/95 benzine spirit. The compound is subsequently homogenized on a roller bed for 24.

The resulting resin-blended polyacrylate PSA is coated onto an etched PET film, with a coat weight of 50 g/m², using a coating knife in a laboratory coating unit.

The coated material is dried at room temperature for 24 hours and subjected to color measurement as a reference.

Subsequently the reference material is heat-treated at 150° C. in a force-air drying oven for 3 hours.

The color and the gloss of the differently stored adhesive tapes and also of the unstored adhesive tape were measured using a BYK Gardner spectro-guide colorimeter. The colors were determined on the CIELab scale with the illuminant D65/10°.

The definitions of the colors in the three-dimensional color space in question are as follows:
L*=lightness (0=black, 100=white)
a*=red-green (−120=green, +120=red)
b*=yellow-blue (−120=blue, +120=yellow)

For each sample, 3 measurements were performed at 3 different locations.

The reference used was a white tile (V+B Steinzeug, article No. 1106 TW01, color 45×). The specimens were subsequently adhered to a reference tile and subjected to measurement.

### Results:

Colorimetry

| Specimen | Thermal load | L* | a* | b* |
|---|---|---|---|---|
| Polyacrylate adhesive | none | 30.21 | −0.76 | 9.61 |
| of Example 2 | | | | |
| Polyacrylate adhesive | 3 hours at 150° C. | 89.81 | −0.3 | 10.51 |
| of Example 2 | | | | |
| Polyacrylate adhesive | 3 hours at 150° C. | 87.62 | −0.52 | 20 |
| of Example 2 without | | | | |
| aging inhibitor | | | | |

### Example 3

A polyacrylate PSA prepared by radical polymerization in 60/95 benzine spirit and consisting of a copolymer based on 48.5 mol % of 2-ethylhexyl acrylate, 48.5 mol % of butyl acrylate, 1 mol % of acrylic acid, and 2 mol % of glycidyl methacrylate, having a weight-average molar mass (M_{w}) of about 1.5 million g/mol, is compounded with about 30% by weight of the terpene-phenolic resin TP95 from Arizona. The compound is subsequently admixed with a mixture of 1% by weight of Irganox 1726 and 1% by weight of Tinuvin 765 in solution in 60/95 benzine spirit. The compound is subsequently homogenized on a roller bed for 24.

The resulting resin-blended polyacrylate PSA is coated onto an etched PET film, with a coat weight of 50 g/m², using a coating knife in a laboratory coating unit.

The coated material is dried at room temperature for 24 hours and subjected to color measurement as a reference.

Subsequently the reference material is heat-treated at 150° C. in a force-air drying oven for 3 hours.

The color and the gloss of the differently stored adhesive tapes and also of the unstored adhesive tape were measured using a BYK Gardner spectro-guide colorimeter. The colors were determined on the CIELab scale with the illuminant D65/10°.

The definitions of the colors in the three-dimensional color space in question are as follows:
L*=lightness (0=black, 100=white)
a*=red-green (−120=green, +120=red)
b*=yellow-blue (−120=blue, +120=yellow)

For each sample, 3 measurements were performed at 3 different locations.

The reference used was a white tile (V+B Steinzeug, article No. 1106 TW01, color 45×). The specimens were subsequently adhered to a reference tile and subjected to measurement.

### Results:

Colorimetry

| Specimen | Thermal load | L* | a* | b* |
|---|---|---|---|---|
| Polyacrylate adhesive | none | 30.21 | −0.76 | 9.61 |
| of Example 3 | | | | |
| Polyacrylate adhesive | 3 hours at 150° C. | 89.81 | −0.3 | 12.31 |
| of Example 3 | | | | |
| Polyacrylate adhesive | 3 hours at 150° C. | 87.62 | −0.52 | 20 |
| of Example 3 without | | | | |
| aging inhibitor | | | | |

### Example 4

An acrylate scaffold polymer prepared by radical solution polymerization and based on 45 mol % of 2-ethylhexyl acrylate, 45 mol % of n-butyl acrylate, 8 mol % of methyl acrylate, 1 mol % of acrylic acid and 1 mol % of 2-hydroxyethyl methacrylate, having a K value of 62, a weight-average molar mass (M_{w}) of 605 000 g/mol, and a polydispersity of 8.6 (solids content approximately 55%), was freed very largely from the solvent by means of a Berstoff single-screw extruder (concentrating extruder). The exit temperature was 105° C., the solids content 99.7%.

The acrylate hotmelt PSA was subsequently introduced into a downstream Welding twin-screw extruder and a tackifier resin and an aging inhibitor were metered in via a solids metering system. The admixed tackifier resin was the terpene-phenolic resin TP95 (Arizona Chemicals), and had an R&B of 95° C. The antioxidant used was Irganox 1726 (4,6-bis(dodecylthiomethyl)-o-cresol) from Ciba.

The compound produced in this way consists of 70 parts by weight of acrylate scaffold polymer, 30 parts by weight of tackifier resin, and 1 part by weight of aging inhibitor.

The acrylate hotmelt PSA was subsequently melted in a feeder extruder and introduced as a polymer melt into a twin-screw extruder.

The crosslinker was added using a suitable metering device to the PSA in the twin-screw extruder. The addition was of 0.45% by weight (based on the acrylate copolymer) of the trimerized HDI polyisocyanate Desmodur N3600 (Bayer AG).

Following emergence of the blended and crosslinked PSA from the twin-screw extruder, it was coated with a coat weight of 50 g/m² onto a pre-coated 23 μm PET film, using a 2-roll applicator unit.

The coated carrier material was subsequently conditioned at room temperature for three days.

Conditioning is followed by thermal loading, in order to determine the discoloration tendency of the acrylate hotmelt adhesive at elevated temperatures:
10 minutes at 140° C. in a drying oven
20 minutes at 140° C. in a drying oven
3 hours at 140° C. in a drying oven

The color and the gloss of the differently stored adhesive tapes and also of the unstored adhesive tape were measured using a BYK Gardner spectro-guide colorimeter. The colors were determined on the CIELab scale with the illuminant D65/10°.

The definitions of the colors in the three-dimensional color space in question are as follows:
L*=lightness (0=black, 100=white)
a*=red-green (−120=green, +120=red)
b*=yellow-blue (−120=blue, +120=yellow)

For each sample, 3 measurements were performed at 3 different locations.

The reference used was a white tile (V+B Steinzeug, article No. 1106 TW01, color 45×). The specimens were subsequently adhered to a reference tile and subjected to measurement.

### Results:

Colorimetry

| Specimen | Thermal load | L* | a* | b* |
|---|---|---|---|---|
| Polyacrylate adhesive | none | 89.93 | −0.04 | 7.67 |
| of Example 4 | | | | |
| Polyacrylate adhesive | 10 minutes at 140° C. | 90.32 | 0.17 | 6.76 |
| of Example 4 | | | | |
| Polyacrylate adhesive | 3 hours at 140° C. | 89.42 | −0.61 | 11.68 |
| of Example 4 | | | | |

### Supplementary Adhesive Performance Testing

Reference specimen after 3-day conditioning, without thermal loading.

| | Bond | Bond | |
|---|---|---|---|
| | strength | strength | Holding power |
| | to steel | to PE | (1 kg/260 mm²) |
| Specimen | [N/cm] | [N/cm] | [min] |
| Polyacrylate adhesive | 7.6 | 3.6 | min 10 000 |
| of Example 1 | | | |

1. A polyacrylate-based pressure-sensitive adhesive comprising at least one resin and at least one ortho-, meta- or para-cresol derivative whose aromatic ring comprises substituents on at least two carbon atoms, the substituents constituting derivatives of thiols and/or thioethers (“sulfur-containing substituents”).

2. The pressure-sensitive adhesive of claim 1, wherein the substituents of the cresol derivative are selected from the group consisting of—S—R and—(CH₂)ₓ—S—R, where x represents a whole number and the radicals R independently of one another represent organic radicals.

3. The pressure-sensitive adhesive of claim 1 wherein the cresol derivative is an ortho-cresol derivative.

4. The pressure-sensitive adhesive of claim 1 wherein the at least two sulfur-containing substituents of the cresol derivative are selected identically.

5. The pressure-sensitive adhesive of claim 1 wherein the at least one cresol derivative represents a compound of the general formula 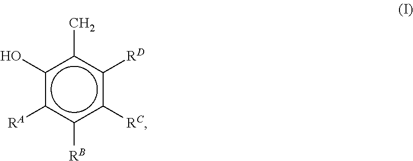
where two of the radicals R^{A}, R^{B}, R^{C}, and R^{D} are selected from the group G1 and the other two radicals R^{A}, R^{B}, R^{C}, and R^{D} are selected independently of one another from the group comprising branched and unbranched alkyl chains and hydrogen.

6. The pressure-sensitive adhesive of claim 1 wherein the sulfur-containing substituents of the cresol derivative are in ortho- and meta-position to the hydroxyl group.

7. The pressure-sensitive adhesive of claim 2 wherein R⁵ and R⁶ are selected independently of one another from the group comprising branched and unbranched saturated hydrocarbon radicals.

8. The pressure-sensitive adhesive of claim 2 wherein x represents a whole number between 1 and 10.

9. The pressure-sensitive adhesive of claim 5 wherein the number of carbon atoms in the unbranched and branched alkyl chains is in the range from 1 to 25.

10. A polyacrylate-based pressure-sensitive adhesive comprising 4,6-bis(alkyl-thioalkyl)-o-cresol.

11. The pressure-sensitive adhesive of claim 1 comprising further sterically hindered phenol derivatives.

12. The pressure-sensitive adhesive of claim 1 further comprising UV absorbers.

13. The pressure-sensitive adhesive of claim 1 wherein the at least one resin is selected from the group consisting of terpene-phenolic resins, rosin derivatives, hydrocarbon resins with aromatic fractions, polyterpene resins, aromatically modified polyterpene resins, and mixtures thereof.

14. A single- or double-sided adhesive tape comprising at least one layer of a pressure-sensitive adhesive according to claim 1.

15. The adhesive tape of claim 14, wherein the at least one layer of pressure-sensitive adhesive has a mass per unit area in the range from 1 to 1000 g/m².

16. The adhesive tape of claim 15 wherein the at least one layer of pressure-sensitive adhesive has a mass per unit area in the range of from 10 to 300 g/m².

17. The pressure-sensitive adhesive of claim 10 comprising a compound selected from the group consisting of 4,6-bis(dodecylthiomethyl)-o-cresol, 4, 6-bis(decylthiomethyl)-o-cresol, 4,6-bis(nonylthiomethyl)-o-cresol and 4,6-bis(octylthiomethyl)-o-cresol.

18. The pressure-sensitive adhesive of claim 8 wherein x is a whole number between 1 and 4.

19. The pressure-sensitive adhesive of claim 9 wherein the number of carbon atoms in the unbranched and branched alkyl chains is in the range of from 8 to 12.
